SEQUENCE LISTING (15 sequences)

SEQ ID NO: 1 - protein, 486 aa, Streptococcus pneumoniae
MSKDIRVRYAPSPTGLLHIGNARTALFNYLYARHHGGTFLIRIEDTDRKRHVEDGERSQLENLRWLGMDWDESPESHENYRQSERLDLYQKYIDQLLAEGKAYKSYVTEEELAAERERQEVAGETPRYINEYLGMSEEEKAAYIAEREAAGIIPTVRLAVNESGIYKWHDMVKGDIEFEGGNIGGDWVIQKKDGYPTYNFAVVIDDHDMQISHVIRGDDHIANTPKQLMVYEALGWEAPEFGHMTLIINSETGKKLSKRDTNTLQFIEDYRKKGYLPEAVFNFIALLGWNPGGEDEIFSREEFIKLFDENRLSKSPAAFDQKKLDWMSNDYIKNADLETIFEMAKPFLEEAGRLTDKAEKLVELYKPQMKSVDEIIPLTDLFFSDFPELTEAEREVMTGETVPTVLEAFKAKLEAMTDDEFVTENIFPQIKAVQKETGIKGKNLFMPIRIAVSGEMHGPELPDTIFLLGREKSIQHIENMLKEISK

SEQ ID NO: 2 - protein, 29 aa, Artificial Sequence
  Synthetic
KNADLETIFEMAKPFLEEAGRLTDKAEKL

SEQ ID NO: 3 - protein, 155 aa, Artificial Sequence
  Synthetic
XKNADLETIFEMAKPFLEEAGRLTDKAEKLVELYKPQMKSVDEIIPLTDLFFSDFPELTEAEREVMTGETVPTVLEAFKAKLEAMTDDKFVTENIFPQIKAVQKETGIKGKNLFMPIRIAVSGEMHGPELPDTIFLLGREKSIQHIENMLKEISK

SEQ ID NO: 4 - protein, 155 aa, Artificial Sequence
  Synthetic
XKNADLETIFEMAKPFLEEAGRLTDKAEKLVELYKPQMKSVDEIIPLTDXFFSDFPELTEAEREVMTXETVPTVLEAFKAKLEAMTDDXFVTENIFPQIKAVQKETGIKGKNLFMPIRIAVSGEMHGPELPDTXFLLGREKSIQHIENXLKEISK

SEQ ID NO: 5 - protein, 36 aa, Artificial Sequence
  Synthetic
XKNADLETIFEMAKPFLEEAGRLTDKAEKLVELYKP

SEQ ID NO: 6 - protein, 55 aa, Artificial Sequence
  Synthetic
KNADLETIFEMAKPFLEEAGRLTDKAEKLVELYKPQMKSVDEIIPLTDLFFSDFP

SEQ ID NO: 7 - protein, 81 aa, Artificial Sequence
  Synthetic
XKNADLETIFEMAKPFLEEAGRLTDKAEKLVELYKPQMKSVDEIIPLTDLFFSDFPELTEAEREVMTGETVPTVLEAFKAK

SEQ ID NO: 8 - protein, 106 aa, Artificial Sequence
  Synthetic
XKNADLETIFEMAKPFLEEAGRLTDKAEKLVELYKPQMKSVDEIIPLTDLFFSDFPELTEAEREVMTGETVPTVLEAFKAKLEAMTDDKFVTENIFPQIKAVQKET

SEQ ID NO: 9 - protein, 123 aa, Artificial Sequence
  Synthetic
XKNADLETIFEMAKPFLEEAGRLTDKAEKLVELYKPQMKSVDEIIPLTDLFFSDFPELTEAEREVMTGETVPTVLEAFKAKLEAMTDDKFVTENIFPQIKAVQKETGIKGKNLFMPIRIAVSG

SEQ ID NO: 10 - protein, 139 aa, Artificial Sequence
  Synthetic
XKNADLETIFEMAKPFLEEAGRLTDKAEKLVELYKPQMKSVDEIIPLTDLFFSDFPELTEAEREVMTGETVPTVLEAFKAKLEAMTDDKFVTENIFPQIKAVQKETGIKGKNLFMPIRIAVSGEMHGPELPDTIFLLGR

SEQ ID NO: 11 - DNA, 465 bp, Artificial Sequence
  Recombinant
aagaatgcagaccttgaaaccatctttgaaatggcaaaaccattcttagaggaagcaggccgtttgactgacaaggctgaaaaattagttgagctctataaaccacaaatgaaatcagtagatgagattatcccattgacagatcttttcttctcagatttcccagaattgacagaagcagagcgcgaagtcatgacgggtgaaacagttccaacagttcttgaagcattcaaagcaaaacttgaagcgatgacagatgataaatttgtgacagaaaatatcttcccacaaattaaagcagttcaaaaagaaacaggtattaaagggaaaaatcttttcatgcctattcgtatcgcagtttcaggcgaaatgcatgggccagaattaccagatacaattttcttgcttggacgtgaaaaatcaattcagcatatcgaaaacatgctaaaagaaatctctaaataa

SEQ ID NO: 12 - protein, 523 aa, Homo sapiens
MAALLRRLLQRERPSAASGRPVGRREANLGTDAGVAVRVRFAPSPTGFLHLGGLRTALYNYIFAKKYQGSFILRLEDTDQTRVVPGAAENIEDMLEWAGIPPDESPRRGGPAGPYQQSQRLELYAQATEALLKTGAAYPCFCSPQRLELLKKEALRNHQTPRYDNRCRNMSQEQVAQKLAKDPKPAIRFRLEQVVPAFQDLVYGWNRHEVASVEGDPVIMKSDGFPTYHLACVVDDHHMGISHVLRGSEWLVSTAKHLLLYQALGWQPPHFAHLPLLLNRDGSKLSKRQGDVFLEHFAADGFLPDSLLDIITNCGSGFAENQMGRTLPELITQFNLTQVTCHSALLDLEKLPEFNRLHLQRLVSNESQRRQLVGKLQVLVEEAFGCQLQNRDVLNPVYVERILLLRQGHICRLQDLVSPVYSYLWTRPAVGRAQLDAISEKVDVIAKRVLGLLERSSMSLTQDMLNGELKKLSEGLEGTKYSNVMKLLRMALSGQQQGPPVAEMMLALGPKEVRERIQKVVSS

SEQ ID NO: 13 - DNA, 28 bp, Artificial Sequence
  Primer
ggaattcaagaatgcagaccttgaaacc

SEQ ID NO: 14 - DNA, 33 bp, Artificial Sequence
  Primer
ccgctcgagttatttagagatttcttttagcat

SEQ ID NO: 15 - DNA, 468 bp, Artificial Sequence
  Artificial recombinant
atgaaaaacgctgatctggaaactatttttgaaatggcaaaaccgtttctggaagaagcaggtcgtctgactgacaaagcagagaaactggttgagctgtacaaaccgcagatgaaatctgttgacgagatcattccgctgactgacctgttcttttctgatttcccggaactgactgaagcagaacgtgaagtaatgactggtgaaactgttccgactgttctggaagcgttcaaagctaaactggaggctatgaccgacgataaattcgtcaccgaaaacatctttccgcagatcaaagcggttcagaaagaaaccggtatcaaaggcaaaaacctgttcatgccgattcgtattgcagtatctggtgaaatgcatggtccggaactgccggatactatctttctgctgggtcgtgagaaatctatccagcacattgagaacatgctgaaagagatctccaaataa

(19) United States
(12) Patent Application Publication
(10) Pub. No.: US 2011/0236470 A1
(43) Pub. Date: Sep. 29, 2011

(54) GLUTAMYL tRNA SYNTHETASE (GtS) FRAGMENTS

(76) Inventors: Yaffa Mizrachi Nebenzahl, Beer Sheva (IL); Michael Tal, Kefar Bilu (IL); Ron Dagan, Omer (IL)

(21) Appl. No.: 13/131,595

(22) PCT Filed: Dec. 3, 2009
(86) PCT No.: PCT/IL2009/001142

§ 371 Date: May 26, 2011

Related U.S. Application Data
(60) Provisional application No. 61/119,383, filed on Dec. 3, 2008.

(51) Int. Cl. A61K 9/127; C12N 9/00; C07H 21/04; A61K 39/09; A61P 37/00

(52) U.S. Cl. 424/450; 435/183; 536/23.2; 424/190.1

(57) ABSTRACT

The present invention relates to polypeptide fragments, including variants and analogs, of Streptococcus pneumonia (S. pneumoniae) glutamyl tRNA synthetase (GtS) protein and to vaccines that include such polypeptide fragments. In particular, the present invention relates to the use of such vaccines for eliciting protective immunity to S. pneumoniae.

### FIELD OF THE INVENTION

The present invention relates to the protein glutamyl tRNA synthetase (GtS) derived from Streptococcus pneumonia (S. pneumoniae) cell wall. In particular, the present invention relates to immunogenic fragments of GtS and to their use as polypeptide-based vaccines eliciting protective immunity against S. pneumoniae.

### BACKGROUND OF THE INVENTION

Streptococcus pneumoniae belongs to the commensal flora of the human respiratory tract, but can also cause invasive infections such as meningitis and sepsis. Most children in the developing world become nasopharyngeal carriers of Streptococcus pneumoniae. Many develop pneumococcal disease that can be invasive (such as bacteremia, sepsis or meningitis), or mucosal infections (such as pneumonia and otitis media). S. pneumoniae is the leading cause of non-epidemic childhood meningitis in Africa and other regions of the developing world. Approximately, one to two million children die from pneumococcal pneumonia each year. Specifically, when considering deaths of children under five years old worldwide, about 20% is from pneumococcal pneumonia. These high morbidity and mortality rates and the persistent emergence of antibiotic resistant strains of S. pneumoniae heighten the need to develop an effective means of prevention, such as vaccination. The pneumococcal 7-valent polysaccharide conjugate vaccine reduced significantly the rates of invasive diseases in infants and restricted significantly the rates of invasive diseases in the non vaccinated members of the community (Kyaw et al., N. Engl. J. Med. 2006, 354, 1455-63). However, carriage and diseases resulting from strains not included in the vaccine are on the rise (Musher D M., N. Engl. J. Med. 2006, 354, 1522-4, Huang et al., Pediatrics 2005, 116, e408-13).

An optimal anti-pneumococcal vaccine should be safe, efficacious, wide-spectrum (covering most pneumococcal strains) and affordable (cheap and available in large quantities). The existing pneumococcal polysaccharide and polysaccharide-conjugated vaccines protect against a narrow but significant group of pneumococcal serotypes, vaccinated subjects remaining susceptible to strains not covered by the vaccines. Of note, the current pneumococcal conjugate vaccines generally have lower coverage against pneumococcal strains causing disease in the developing world compared to developed countries. In addition to limitations of coverage, conjugate vaccines are complex to produce and expensive, resulting in restricted quantities and are beyond the budget of many poor countries.

The mucosal epithelial surfaces with their tight junctions constitute the first line of defense that prevents the entry of pathogens and their products. S. pneumoniae adhere to the nasopharyngeal mucosal cells causing carriage without an overt inflammatory response. For clinical disease to occur, S. pneumoniae have to spread from the nasopharynx into the middle ear or the lungs or cross the mucosal epithelial cell layer and be deposited basally within the submucosa (Ring et al., J. Clin. Invest. 1998, 102:347-60). Molecules involved in adhesion, spread and invasion of S. pneumoniae, include capsular polysaccharides, cell-wall peptidoglycan and surface proteins (Jedrzejas M J. Microbiol. Mol. Biol. Rev. 2001, 65, 187-207).

It has been observed that in infants that the antibody response to S. pneumoniae cell wall proteins increases with age and correlates negatively with morbidity (Lifshitz et al. Clin. Exp. Immunol. 2002, 127, 344-53). A longitudinal series of children's sera was utilized to identify S. pneumoniae cell wall proteins that exhibit age-dependent antigenicity (Ling et al., Clin Exp Immunol 2004, 138, 290-8), using biochemical, immunological and MALDI TOF studies. One such protein is Glutamyl tRNA Synthetase (GtS).

Mizrachi-Nebenzahl et al. 2007 (J. Infect. Dis., 196, 945-53), discloses that Streptococcus pneumoniae derived recombinant GtS, is able to induce a partially protective immune response in mice.

International Patent Application Publication No. WO 02/077021, assigned to Chiron S. P. A., discloses the sequence of about 2,500 S. pneumoniae type 4 strain genes, including the GtS gene, and their corresponding amino acid sequences that were identified in silico. The use of a subset of 432 of those protein sequences as antigens for immunization is also suggested although no working examples for the use of the proteins as antigens in the production of vaccines are provided.

International Patent Application Publication No. WO 97/38718 assigned to SmithKline Beecham Corp. discloses S. pneumoniae GtS polypeptides of 480, 348, 126 and 62 amino acids, polynucleotides encoding the GtS polypeptides and methods for producing such polypeptides by recombinant techniques. Also provided are vaccine formulations comprising GtS polypeptides although no such vaccine was actually prepared at the time of filing. U.S. Pat. No. 5,958,734 claims GtS N-terminus fragment of 348 and C-terminus 126 amino acids fragment. U.S. Pat. No. 5,976,840 claims a 480 amino acids GtS sequence starting at Val-7, and variants containing up to three nucleotide substitutions, deletions, or nucleotide insertions for every 100 nucleotides. U.S. Pat. No. 6,300,119 claims a GtS variant polynucleotide comprising a sequence identical to the polynucleotide encoding the above 480 amino acids polypeptide, except that up to five nucleotides may be substituted, deleted or inserted for every 100 nucleotides, and wherein the first polynucleotide sequence detects Streptococcus pneumoniae by hybridization. U.S. Pat. No. 6,165,760 relates to the GtS polypeptide the above of 480 amino acids sequence further comprising a heterologous amino acid sequence.

WO 03/082183 to one of the inventors of the present application discloses a defined group of cell wall and cell membrane S. pneumoniae proteins for use as vaccines against said bacteria. The thirty eight identified S. pneumoniae proteins, including the intact GtS, were found to have age dependent immunogenicity in children attending day care centers.

There is an unmet need for an improved S. pneumoniae polypeptide-based vaccine which can induce long-lasting immunological responses, having broad specificity against a wide range of different S. pneumoniae serotypes, and in all age groups, including young children and elderly people. There is also a need for a vaccine based on a polypeptide sequence having minimal homology with human proteins.

### SUMMARY OF THE INVENTION

The present invention provides immunogenic glutamyl tRNA synthetase (GtS) protein fragments and vaccines against S. pneumoniae. The polypeptides of the present invention which are fragments of the S. pneumoniae protein GtS, were selected to possess reduced homology to human sequences compared to the intact protein, minimizing the risk of developing antibodies against the immunized subject own proteins. Furthermore, the polypeptides of the present invention have high sequence identity among S. pneumoniae strains currently sequenced making them ideal for developing wide-spectrum vaccines against the bacterium. It was surprisingly found that GtS fragments of the invention are more active than the intact protein in eliciting an immune response against S. pneumoniae.

According to the present invention the GtS fragments can be produced recombinantly, as isolated polypeptides or as a fusion protein, or synthetically by peptide synthesis or by linking shorter synthetic peptide fragments. Recombinant or synthetic production can be used, according to the present invention, to introduce specific mutations and/or variations in the polypeptide fragment sequence for improving specific properties such as solubility and stability.

A polypeptide fragment, shorter than the intact protein, provides more immunogenic epitopes per microgram of protein.

The polypeptides of the present invention can be used in vaccines against S. pneumoniae alone, as part of a chimeric protein, which may be used as an adjuvant, or mixed or formulated with an external adjuvant.

According to one aspect the present invention provides a synthetic or recombinant polypeptide of 50-250 amino acids derived from the sequence of S. pneumoniae GtS (SEQ ID NO:1), comprising the sequence KNADLETIFEMAKPFLEEAGRLTDKAEKL (SEQ ID NO:2), and variants and analogs thereof.

Variants include substitution of one amino acid residue per each ten amino acid residues in a polypeptide sequence, namely, polypeptides having 90% or more identity are included within the scope of the present invention. According to some embodiments, sequences having at least 97% identity to the polypeptides of the present invention are provided.

According to some embodiments the polypeptide consists of 100-200 amino acids. According to other embodiments, the polypeptide consists of about 130-180 amino acids.

According to some embodiments, the GtS polypeptide according to the invention share less than about 24% sequence identity with the human GtS-2 protein of SEQ ID NO:12. According to other embodiments, the GtS polypeptide according to the invention share less than about 10% sequence identity with the human GtS-2 protein of SEQ ID NO:12. According to some embodiments, the GtS polypeptide of to the invention share less than about 18% sequence identity with residues 361-521 of SEQ ID NO:12. According to yet another embodiment, when aligning the sequence of a GtS polypeptide according to the invention with the sequence of human GtS-2 (SEQ ID NO:12), no more than six contiguous amino acid residues are identical between the two sequences.

According to some embodiments the present invention provides a synthetic or recombinant GtS polypeptide fragment comprising the sequence: XKNADLETIFEMAKPFLEEAGRLTDKAEKLVELYKPQMKSVDEIIPLTDLFFSDFP ELTEAEREVMTGETVPTVLEAFKAKLEAMTDDKFVTENIFPQIKAVQKETGIKGK NLFMPIRIAVSGEMHGPELPDTIFLLGREKSIQHIENMLKEISK (SEQ ID NO:3, residues 333-486 of SEQ ID NO:1), wherein X is Methionine or represents the polypeptide's N-terminus, and variants and analogs thereof.

According to other embodiments the synthetic or recombinant GtS polypeptide fragment comprises the sequence:
XKNADLETIFEMAKPFLEEAGRLTDKAEKLVELYKPQMKSVDEIIPLT DX₁FFSDFPELTEAEREVMTX₂ETVPTVLEAFKAKLEAMTDDX₃FVTEN IFPQIKAVQKETGIKGKNLFMPIRIAVSGEMHGPELPDTX₄FLLGREKSI QHIENX₅LKEISK (SEQ ID NO: 4), wherein X is Methionine or represents the polypeptide's N-terminus, X₁ is Leu (L) or Fhe (F), X₂ is Gly (G) or Asp (D), X₃ is Lys (K) or Glu (E), X₄ is Ile (I) or Val (V), and X₅ is Met (M) or Ile (I), and variants and analogs thereof.

According to yet other embodiments the synthetic or recombinant GtS polypeptide fragment comprises a sequence selected from the group consisting of:

(SEQ ID NO: 5)
XKNADLETIFEMAKPFLEEAGRLTDKAEKLVELYKP;
(SEQ ID NO: 6)
KNADLETIFEMAKPFLEEAGRLTDKAEKLVELYKPQMKSVDEIIPLTD
LFFSDFP;
(SEQ ID NO: 7)
XKNADLETIFEMAKPFLEEAGRLTDKAEKLVELYKPQMKSVDEIIPLT
DLFFSDFPELTEAEREVMTGETVPTVLEAFKAK;
(SEQ ID NO: 8)
XKNADLETIFEMAKPFLEEAGRLTDKAEKLVELYKPQMKSVDEIIPLT
DLFFSDFPELTEAEREVMTGETVPTVLEAFKAKLEAMTDDKFVTENIF
PQIKAVQKET
(SEQ ID NO: 9)
XKNADLETIFEMAKPFLEEAGRLTDKAEKLVELYKPQMKSVDEIIPLT
DLFFSDFPELTEAEREVMTGETVPTVLEAFKAKLEAMTDDKFVTENIF
PQIKAVQKETGIKGKNLFMPIRIAVSG;
and
(SEQ ID NO: 10)
XKNADLETIFEMAKPFLEEAGRLTDKAEKLVELYKPQMKSVDEIIPLT
DLFFSDFPELTEAEREVMTGETVPTVLEAFKAKLEAMTDDKFVTENIF
PQIKAVQKETGIKGKNLFMPIRIAVSGEMHGPELPDTIFLLGR,

wherein X is Methionine or represents the polypeptide's N-terminus,
and variants and analogs thereof.

According to yet other embodiments the present invention provides a synthetic or recombinant GtS polypeptide fragment consisting of a sequence selected from the group of SEQ ID NO:3 to SEQ ID NO:10.

According to some embodiments the polypeptide fragments are not conjugated or fused to a carrier protein. In other embodiments the polypeptide fragments of the present invention are produced as a recombinant fusion protein comprising a carrier sequence, namely the fragments are inserted within a sequence of a carrier polypeptide or are fused to an amino terminal, carboxy terminal or side chain of a carrier protein sequence, or to another S. pneumoniae protein or polypeptide).

The present invention provides, according to another aspect, isolated polynucleotide sequences encoding the GtS fragment polypeptides.

According to some embodiments the isolated polynucleotide sequences encode a polypeptide sequence of 50-250 amino acids comprising the sequence KNADLETIFEMAKPFLEEAGRLTDKAEKL (SEQ ID NO:2), and variants and analogs thereof. According to some preferred embodiments the isolated polynucleotide sequences encode a polypeptide sequence consisting of 100-200 amino acids.

According to some specific embodiments the isolated polynucleotide sequence comprises SEQ ID NO:11 or SEQ ID NO:15. According to some specific embodiments the isolated polynucleotide sequence consists of SEQ ID NO:11 or SEQ ID NO:15.

According to additional embodiments the isolated polynucleotide sequence encode a polypeptide sequence selected from the group consisting of: SEQ ID NO:3 to SEQ ID NO:10, and variants and analogs thereof.

According to yet another aspect, the present invention provides vaccine compositions for immunization of a subject against S. pneumoniae comprising at least one synthetic or recombinant GtS polypeptide fragment of 50-250 amino acids comprising the sequence KNADLETIFEMAKPFLEEAGRLTDKAEKL (SEQ ID NO:2), and variants and analogs thereof. According to some preferred embodiments the polypeptide consists of 100-200 amino acids.

According to some embodiments the vaccine composition comprises a GtS polypeptide sequence selected from the group consisting of: SEQ ID NO:3 to SEQ ID NO:10, and variants and analogs thereof.

According to other embodiments, a vaccine composition according to the present invention further comprises at least one additional S. pneumoniae polypeptide or protein sequence.

According to some embodiments the vaccine composition according to the present invention further comprises an adjuvant. According to other embodiments the vaccine does not contain an adjuvant.

Pharmaceutically acceptable adjuvants include, but are not limited to water in oil emulsions, lipid emulsions, and liposomes. According to some embodiments the adjuvant is selected from the group consisting of: Montanide®, alum, muramyl dipeptide, Gelvac®, chitin microparticles, chitosan, cholera toxin subunit B, labile toxin, AS21A, Intralipid®, and Lipofundin®.

In some embodiments the vaccine is formulated for intramuscular, intranasal, oral, intraperitoneal, subcutaneous, topical, intradermal and transdermal delivery. In some embodiments the vaccine is formulated for intramuscular administration. In other embodiments the vaccine is formulated for oral administration. In yet other embodiments the vaccine is formulated for intranasal administration.

The present invention provides according to a further embodiment a method for inducing an immune response and conferring protection against S. pneumoniae in a subject, comprising administering a vaccine composition comprising at least one synthetic or recombinant GtS polypeptide fragment of 50-250 amino acids comprising the sequence KNADLETIFEMAKPFLEEAGRLTDKAEKL (SEQ ID NO:2), and variants and analogs thereof. According to some preferred embodiments the polypeptide consists of 100-200 amino acids.

Any route of administration can be utilized to deliver the vaccines of the present invention. According to some embodiments, the route of administration of the vaccine is selected from intramuscular, oral, intranasal, intraperitoneal, subcutaneous, topical, intradermal, and transdermal delivery. According to some embodiments the vaccine is administered by intramuscular, intranasal or oral routs.

According to a further aspect of the present invention, synthetic or recombinant GtS polypeptide fragment of 50-250 amino acids comprising the sequence KNADLETIFEMAKPFLEEAGRLTDKAEKL (SEQ ID NO:2), and variants and analogs thereof, are used for prevention of S. pneumoniae infection in a subject. According to some preferred embodiments the polypeptide consists of 100-200 amino acids.

Use of a polypeptide according to the invention for preparation of a vaccine composition for immunization against S. pneumoniae is also within the scope of the present invention, as well as use of an isolated polynucleotide according to the invention for production of a GtS polypeptide fragment of 50-250 amino acids comprising the sequence KNADLETIFEMAKPFLEEAGRLTDKAEKL (SEQ ID NO:2), and variants and analogs thereof. According to some preferred embodiments the polypeptide consists of 100-200 amino acids.

All the polypeptides disclosed in the present invention can be produced by recombinant methods and by chemical synthesis.

Another aspect of the present invention provides a fusion protein comprising at least one GtS fragment polypeptide and at least one additional polypeptide sequence.

According to one embodiment the fusion protein comprises a GtS polypeptide fragment of 100-200 amino acids comprising the sequence KNADLETIFEMAKPFLEEAGRLTDKAEKL (SEQ ID NO:2), and variants and analogs thereof.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 shows PCR amplification by genomic DNA of the GtS fragment (333-486).

FIG. 2 depicts a gel confirming of the existence of the expected 462 bp insert by PCR amplification.

FIG. 3 represents resolution of the eluted GtS fragment 333-486 (23 kDa band) by 1D-PAGE stained with Coomassie Brilliant Blue.

FIG. 4 shows western blot analysis of the recombinant GtS fragment 333-486 HIS-tagged fusion protein (23 kDa band) by 1D-PAGE using anti-HIS-tagged antibodies.

FIG. 5 demonstrates survival of mice challenged with S. pneumoniae neutralized ex-vivo with rabbit anti GtS₃₃₃₋₄₈₆ fragment.

FIG. 6 SDS-PAGE Coomassie stained of untagged GtS 333-486 fragment (sGtS) obtained from three consecutive tubes collected from the first G-200 preparative column cycle.

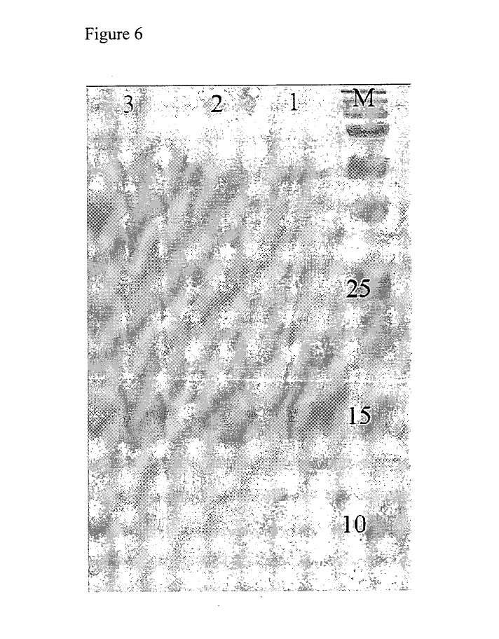

### DETAILED DESCRIPTION OF THE INVENTION

The present invention provides polypeptides derived from the sequence of S. pneumoniae GtS protein, and vaccines containing these polypeptides. A polypeptide according to the present invention comprises the 29 amino acid residues KNADLETIFEMAKPFLEEAGRLTDKAEKL (SEQ ID NO:2) corresponding to residues 333-361 of the intact S. pneumoniae GtS protein of SEQ ID NO:1.

The polypeptides of the present invention have the advantage of reduced homology to human sequences. If a microbial antigen has significant sequence homology to a human protein, then use of such an antigen in a vaccine would entail the risk of eliciting antibodies directed against the particular human protein, with resultant risk of auto-immunity—an unacceptable outcome. Therefore, it is very important to remove any such sequences—homologous between the microbial antigen and the human protein—from the antigen in order that it would have utility as a vaccine antigen.

A polypeptide fragment of 154 amino acids corresponding to amino acid residues 333-486 of the S. pneumoniae GtS protein was produced, characterized and found to be effective in producing neutralizing antibodies in rabbits against S. pneumoniae infection. Surprisingly, the 154 amino acids GtS fragment was found to be more effective than the corresponding intact protein in neutralizing the infectious bacterium.

For convenience, certain terms employed in the specification, examples and claims are described herein.

The term “antigen presentation” means the expression of antigen on the surface of a cell in association with major histocompatibility complex class I or class II molecules (MHC-I or MHC-II) of animals or with the HLA-I and HLA-II of humans.

The term “immunogenicity” or “immunogenic” relates to the ability of a substance to stimulate or elicit an immune response. Immunogenicity is measured, for example, by determining the ability to produce antibodies specific for the substance. The presence of antibodies is detected by methods known in the art, for example using an ELISA assay.

“Amino acid sequence”, as used herein, refers to an oligopeptide, peptide, polypeptide, or protein sequence, and fragment thereof, and to naturally occurring or synthetic molecules.

A “chimeric protein” or “fusion protein” are used interchangeably and refer to a polypeptide operatively linked to a polypeptide other than the polypeptide from which the GtS polypeptide fragment was derived.

### Recombinant Production of Polypeptides

The polypeptide fragments of the present invention can be prepared by expression in an expression vector per se or as a chimeric protein. The methods to produce a chimeric or recombinant protein comprising one or more GtS polypeptide fragment are known to those with skill in the art. A nucleic acid sequence encoding one or more GtS polypeptide fragment can be inserted into an expression vector for preparation of a polynucleotide construct for propagation and expression in host cells.

The term “expression vector” and “recombinant expression vector” as used herein refers to a DNA molecule, for example a plasmid or virus, containing a desired and appropriate nucleic acid sequences necessary for the expression of the recombinant polypeptides for expression in a particular host cell. As used herein “operably linked” refers to a functional linkage of at least two sequences. Operably linked includes linkage between a promoter and a second sequence, for example an nucleic acid of the present invention, wherein the promoter sequence initiates and mediates transcription of the DNA sequence corresponding to the second sequence.

The regulatory regions necessary for transcription of the polypeptides can be provided by the expression vector. The precise nature of the regulatory regions needed for gene expression may vary among vectors and host cells. Generally, a promoter is required which is capable of binding RNA polymerase and promoting the transcription of an operably-associated nucleic acid sequence. Regulatory regions may include those 5′ non-coding sequences involved with initiation of transcription and translation, such as the TATA box, capping sequence, CAAT sequence, and the like. The non-coding region 3′ to the coding sequence may contain transcriptional termination regulatory sequences, such as terminators and polyadenylation sites. A translation initiation codon (ATG) may also be provided.

In order to clone the nucleic acid sequences into the cloning site of a vector, linkers or adapters providing the appropriate compatible restriction sites are added during synthesis of the nucleic acids. For example, a desired restriction enzyme site can be introduced into a fragment of DNA by amplification of the DNA by use of PCR with primers containing the desired restriction enzyme site.

An alternative method to PCR is the use of synthetic gene. The method allows production of an artificial gene which comprise an optimized sequence of nucleotide to be express in desired species (for example E. coli). Redesigning a gene offers a means to improve gene expression in many cases. Rewriting the open reading frame is possible because of the redundancy of the genetic code. Thus it is possible to change up to about a third of the nucleotides in an open reading frame and still produce the same protein. For a typical protein sequence of 300 amino acids there are over 10¹⁵⁰ codon combinations that will encode an identical protein. Using optimization methods such as replacing rarely used codons with more common codons can result in dramatic effects. Further optimizations such as removing RNA secondary structures can also be included. Computer programs are available to perform these and other simultaneous optimizations. A well optimized gene can improve dramatically protein expression. Because of the large number of nucleotide changes made to the original DNA sequence, the only practical way to create the newly designed genes is to use gene synthesis.

An expression construct comprising a GtS polypeptide fragment sequence operably associated with regulatory regions can be directly introduced into appropriate host cells for expression and production of polypeptide per se or as recombinant fusion protein. The expression vectors that may be used include but are not limited to plasmids, cosmids, phage, phagemids or modified viruses. Typically, such expression vectors comprise a functional origin of replication for propagation of the vector in an appropriate host cell, one or more restriction endonuclease sites for insertion of the desired gene sequence, and one or more selection markers.

The recombinant polynucleotide construct comprising the expression vector and a GtS polypeptide fragment should then be transferred into a bacterial host cell where it can replicate and be expressed. This can be accomplished by methods known in the art. The expression vector is used with a compatible prokaryotic or eukaryotic host cell which may be derived from bacteria, yeast, insects, mammals and humans.

Once expressed by the host cell, the GtS polypeptide fragment can be separated from undesired components by a number of protein purification methods. One such method uses a polyhistidine tag on the recombinant protein. A polyhistidine-tag consists in at least six histidine (His) residues added to a recombinant protein, often at the N- or C-terminus. Polyhistidine-tags are often used for affinity purification of polyhistidine-tagged recombinant proteins that are expressed in E. coli or other prokaryotic expression systems. The bacterial cells are harvested by centrifugation and the resulting cell pellet can be lysed by physical means or with detergents or enzymes such as lysozyme. The raw lysate contains at this stage the recombinant protein among several other proteins derived from the bacteria and are incubated with affinity media such as NTA-agarose, HisPur resin or Talon resin. These affinity media contain bound metal ions, either nickel or cobalt to which the polyhistidine-tag binds with micromolar affinity. The resin is then washed with phosphate buffer to remove proteins that do not specifically interact with the cobalt or nickel ion. The washing efficiency can be improved by the addition of 20 mM imidazole and proteins are then usually eluted with 150-300 mM imidazole. The polyhistidine tag may be subsequently removed using restriction enzymes, endoproteases or exoproteases. Kits for the purification of histidine-tagged proteins can be purchased for example from Qiagen.

Another method is through the production of inclusion bodies, which are inactive aggregates of protein that may form when a recombinant polypeptide is expressed in a prokaryote. While the cDNA may properly code for a translatable mRNA, the protein that results may not fold correctly, or the hydrophobicity of the sequence may cause the recombinant polypeptide to become insoluble. Inclusion bodies are easily purified by methods well known in the art. Various procedures for the purification of inclusion bodies are known in the art. In some embodiments the inclusion bodies are recovered from bacterial lysates by centrifugation and are washed with detergents and chelating agents to remove as much bacterial protein as possible from the aggregated recombinant protein. To obtain soluble protein, the washed inclusion bodies are dissolved in denaturing agents and the released protein is then refolded by gradual removal of the denaturing reagents by dilution or dialysis (as described for example in Molecular cloning: a laboratory manual, 3rd edition, Sambrook, J. and Russell, D. W., 2001; CSHL Press).

An analytical purification generally utilizes three properties to separate proteins. First, proteins may be purified according to their isoelectric points by running them through a pH graded gel or an ion exchange column. Second, proteins can be separated according to their size or molecular weight via size exclusion chromatography or by SDS-PAGE (sodium dodecyl sulfate-polyacrylamide gel electrophoresis) analysis. Proteins are often purified by using 2D-PAGE and are then analysed by peptide mass fingerprinting to establish the protein identity. Thirdly, proteins may be separated by polarity/hydrophobicity via high pressure liquid chromatography or reversed-phase chromatography. The purified protein is followed by its molecular mass or other methods known in the art.

In order to evaluate the process of multistep purification, the amount of the specific protein has to be compared to the amount of total protein. The latter can be determined by the Bradford total protein assay or by absorbance of light at 280 nm, however some reagents used during the purification process may interfere with the quantification. For example, imidazole (commonly used for purification of polyhistidine-tagged recombinant proteins) is an amino acid analogue and at low concentrations will interfere with the bicinchoninic acid (BCA) assay for total protein quantification. Impurities in low-grade imidazole will also absorb at 280 nm, resulting in an inaccurate reading of protein concentration from UV absorbance.

Another method to be considered is Surface Plasmon Resonance (SPR). SPR can detect binding of label free molecules on the surface of a chip. If the desired protein is an antibody, binding can be translated to directly to the activity of the protein. One can express the active concentration of the protein as the percent of the total protein. SPR can be a powerful method for quickly determining protein activity and overall yield.

### Vaccine Formulation

The vaccine compositions of the present invention comprise at least one GtS polypeptide fragment, and optionally, an adjuvant. Formulation can contain a variety of additives, such as adjuvant, excipient, stabilizers, buffers, or preservatives. The vaccine can be formulated for administration in one of many different modes.

In some embodiments, the vaccine is formulated for parenteral administration, for example intramuscular administration. According to yet another embodiment the administration is orally. According to some embodiments administration is oral and the vaccine is presented, for example, in the form of a tablet or encased in a gelatin capsule or a microcapsule.

According to yet another embodiment the administration is intradermal. Needles specifically designed to deposit the vaccine intradermally are known in the art as disclosed for example in U.S. Pat. No. 6,843,781 and U.S. Pat. No. 7,250,036 among others. According to other embodiments the administration is performed with a needleless injector.

According to one embodiment of the invention, the vaccine is administered intranasally. The vaccine formulation may be applied to the lymphatic tissue of the nose in any convenient manner. However, it is preferred to apply it as a liquid stream or liquid droplets to the walls of the nasal passage. The intranasal composition can be formulated, for example, in liquid form as nose drops, spray, or suitable for inhalation, as powder, as cream, or as emulsion.

The formulation of these modalities is general knowledge to those with skill in the art.

Liposomes provide another delivery system for antigen delivery and presentation. Liposomes are bilayered vesicles composed of phospholipids and other sterols surrounding a typically aqueous center where antigens or other products can be encapsulated. The liposome structure is highly versatile with many types range in nanometer to micrometer sizes, from about 25 nm to about 500 μm. Liposomes have been found to be effective in delivering therapeutic agents to dermal and mucosal surfaces. Liposomes can be further modified for targeted delivery by for example, incorporating specific antibodies into the surface membrane, or altered to encapsulate bacteria, viruses or parasites. The average survival time or half life of the intact liposome structure can be extended with the inclusion of certain polymers, for example polyethylene glycol, allowing for prolonged release in vivo. Liposomes may be unilamellar or multilamellar.

The vaccine composition may be formulated by: encapsulating an antigen or an antigen/adjuvant complex in liposomes to form liposome-encapsulated antigen and mixing the liposome-encapsulated antigen with a carrier comprising a continuous phase of a hydrophobic substance. If an antigen/adjuvant complex is not used in the first step, a suitable adjuvant may be added to the liposome-encapsulated antigen, to the mixture of liposome-encapsulated antigen and carrier, or to the carrier before the carrier is mixed with the liposome-encapsulated antigen. The order of the process may depend on the type of adjuvant used. Typically, when an adjuvant like alum is used, the adjuvant and the antigen are mixed first to form an antigen/adjuvant complex followed by encapsulation of the antigen/adjuvant complex with liposomes. The resulting liposome-encapsulated antigen is then mixed with the carrier. The term “liposome-encapsulated antigen” may refer to encapsulation of the antigen alone or to the encapsulation of the antigen/adjuvant complex depending on the context. This promotes intimate contact between the adjuvant and the antigen and may, at least in part, account for the immune response when alum is used as the adjuvant. When another is used, the antigen may be first encapsulated in liposomes and the resulting liposome-encapsulated antigen is then mixed into the adjuvant in a hydrophobic substance.

In formulating a vaccine composition that is substantially free of water, antigen or antigen/adjuvant complex is encapsulated with liposomes and mixed with a hydrophobic substance. In formulating a vaccine in an emulsion of water-in-a hydrophobic substance, the antigen or antigen/adjuvant complex is encapsulated with liposomes in an aqueous medium followed by the mixing of the aqueous medium with a hydrophobic substance. In the case of the emulsion, to maintain the hydrophobic substance in the continuous phase, the aqueous medium containing the liposomes may be added in aliquots with mixing to the hydrophobic substance.

In all methods of formulation, the liposome-encapsulated antigen may be freeze-dried before being mixed with the hydrophobic substance or with the aqueous medium as the case may be. In some instances, an antigen/adjuvant complex may be encapsulated by liposomes followed by freeze-drying. In other instances, the antigen may be encapsulated by liposomes followed by the addition of adjuvant then freeze-drying to form a freeze-dried liposome-encapsulated antigen with external adjuvant. In yet another instance, the antigen may be encapsulated by liposomes followed by freeze-drying before the addition of adjuvant. Freeze-drying may promote better interaction between the adjuvant and the antigen resulting in a more efficacious vaccine.

Formulation of the liposome-encapsulated antigen into a hydrophobic substance may also involve the use of an emulsifier to promote more even distribution of the liposomes in the hydrophobic substance. Typical emulsifiers are well-known in the art and include mannide oleate (Arlacel™ A), lecithin, Tween™ 80, Spans™ 20, 80, 83 and 85. The emulsifier is used in an amount effective to promote even distribution of the liposomes. Typically, the volume ratio (v/v) of hydrophobic substance to emulsifier is in the range of about 5:1 to about 15:1.

Microparticles and nanoparticles employ small biodegradable spheres which act as depots for vaccine delivery. The major advantage that polymer microspheres possess over other depot-effecting adjuvants is that they are extremely safe and have been approved by the Food and Drug Administration in the US for use in human medicine as suitable sutures and for use as a biodegradable drug delivery system (Langer R. Science. 1990; 249(4976):1527-33). The rates of copolymer hydrolysis are very well characterized, which in turn allows for the manufacture of microparticles with sustained antigen release over prolonged periods of time (O'Hagen, et al., Vaccine. 1993; 11(9):965-9).

Parenteral administration of microparticles elicits long-lasting immunity, especially if they incorporate prolonged release characteristics. The rate of release can be modulated by the mixture of polymers and their relative molecular weights, which will hydrolyze over varying periods of time. Without wishing to be bound to theory, the formulation of different sized particles (1 μm to 200 μm) may also contribute to long-lasting immunological responses since large particles must be broken down into smaller particles before being available for macrophage uptake. In this manner a single-injection vaccine could be developed by integrating various particle sizes, thereby prolonging antigen presentation and greatly benefiting livestock producers.

In some applications an adjuvant or excipient may be included in the vaccine formulation. Montanide™ (Incomplete Freund's adjuvant) and alum for example, are preferred adjuvants for human use. The choice of the adjuvant will be determined in part by the mode of administration of the vaccine. A preferred mode of administration is intramuscular administration. Another preferred mode of administration is intranasal administration. Non-limiting examples of intranasal adjuvants include chitosan powder, PLA and PLG microspheres, QS-21, AS02A, calcium phosphate nanoparticles (CAP); mCTA/LTB (mutant cholera toxin E112K with pentameric B subunit of heat labile enterotoxin), and detoxified E. Coli derived labile toxin.

The adjuvant used may also be, theoretically, any of the adjuvants known for peptide- or protein-based vaccines. For example: inorganic adjuvants in gel form (aluminium hydroxide/aluminium phosphate, Warren et al., 1986; calcium phosphate, Relyvelt, 1986); bacterial adjuvants such as monophosphoryl lipid A (Ribi, 1984; Baker et al., 1988) and muramyl peptides (Ellouz et al., 1974; Allison and Byars, 1991; Waters et al., 1986); particulate adjuvants such as the so-called ISCOMS (“immunostimulatory complexes”, Mowat and Donachie, 1991; Takahashi et al., 1990; Thapar et al., 1991), liposomes (Mbawuike et al. 1990; Abraham, 1992; Phillips and Emili, 1992; Gregoriadis, 1990) and biodegradable microspheres (Marx et al., 1993); adjuvants based on oil emulsions and emulsifiers such as IFA (“Incomplete Freund's adjuvant” (Stuart-Harris, 1969; Warren et al., 1986), SAF (Allison and Byars, 1991), saponines (such as QS-21; Newman et al., 1992), squalene/squalane (Allison and Byars, 1991); synthetic adjuvants such as non-ionic block copolymers (Hunter et al., 1991), muramyl peptide analogs (Azuma, 1992), synthetic lipid A (Warren et al., 1986; Azuma, 1992), synthetic polynucleotides (Harrington et al., 1978) and polycationic adjuvants (WO 97/30721).

Adjuvants for use with immunogens of the present invention include aluminum or calcium salts (for example hydroxide or phosphate salts). A particularly preferred adjuvant for use herein is an aluminum hydroxide gel such as Alhydrogel™. Calcium phosphate nanoparticles (CAP) is an adjuvant being developed by Biosante, Inc (Lincolnshire, Ill.). The immunogen of interest can be either coated to the outside of particles, or encapsulated inside on the inside [He et al. (November 2000) Clin. Diagn. Lab. Immunol., 7(6):899-903].

Another adjuvant for use with an immunogen of the present invention is an emulsion. A contemplated emulsion can be an oil-in-water emulsion or a water-in-oil emulsion. In addition to the immunogenic chimer protein particles, such emulsions comprise an oil phase of squalene, squalane, peanut oil or the like as are well known, and a dispersing agent. Non-ionic dispersing agents are preferred and such materials include mono- and di-C₁₂-C₂₄-fatty acid esters of sorbitan and mannide such as sorbitan mono-stearate, sorbitan mono-oleate and mannide mono-oleate.

Such emulsions are for example water-in-oil emulsions that comprise squalene, glycerol and a surfactant such as mannide mono-oleate (Arlacel™ A), optionally with squalane, emulsified with the chimer protein particles in an aqueous phase. Alternative components of the oil-phase include alpha-tocopherol, mixed-chain di- and tri-glycerides, and sorbitan esters. Well-known examples of such emulsions include Montanide™ ISA-720, and Montanide™ ISA 703 (Seppic, Castres, France. Other oil-in-water emulsion adjuvants include those disclosed in WO 95/17210 and EP 0 399 843.

The use of small molecule adjuvants is also contemplated herein. One type of small molecule adjuvant useful herein is a 7-substituted-8-oxo- or 8-sulfo-guanosine derivative described in U.S. Pat. No. 4,539,205, U.S. Pat. No. 4,643,992, U.S. Pat. No. 5,011,828 and U.S. Pat. No. 5,093,318. 7-allyl-8-oxoguanosine(loxoribine) has been shown to be particularly effective in inducing an antigen-(immunogen-) specific response.

A useful adjuvant includes monophosphoryl lipid A (MPL®), 3-deacyl monophosphoryl lipid A (3D-MPL®), a well-known adjuvant manufactured by Corixa Corp. of Seattle, formerly Ribi Immunochem, Hamilton, Mont. The adjuvant contains three components extracted from bacteria: monophosphoryl lipid (MPL) A, trehalose dimycolate (TDM) and cell wall skeleton (CWS) (MPL+TDM+CWS) in a 2% squalene/Tween™ 80 emulsion. This adjuvant can be prepared by the methods taught in GB 2122204B.

Other compounds are structurally related to MPL® adjuvant called aminoalkyl glucosamide phosphates (AGPs) such as those available from Corixa Corp under the designation RC-529™ adjuvant {2-[(R)-3-tetra-decanoyloxytetradecanoylamino]-ethyl-2-deoxy-4-O-phosphon- o-3-O—[(R)-3-tetradecanoyloxytetra-decanoyl]-2-[(R)-3-tetra-decanoyloxytet-radecanoyl-amino]-p-D-glucopyranoside triethylammonium salt}. An RC-529 adjuvant is available in a squalene emulsion sold as RC-529SE and in an aqueous formulation as RC-529AF available from Corixa Corp. (see, U.S. Pat. No. 6,355,257 and U.S. Pat. No. 6,303,347; U.S. Pat. No. 6,113,918; and U.S. Publication No. 03-0092643).

Further contemplated adjuvants include synthetic oligonucleotide adjuvants containing the CpG nucleotide motif one or more times (plus flanking sequences) available from Coley Pharmaceutical Group. The adjuvant designated QS21, available from Aquila Biopharmaceuticals, Inc., is an immunologically active saponin fractions having adjuvant activity derived from the bark of the South American tree Quillaja Saponaria Molina (e.g. Quil™ A), and the method of its production is disclosed in U.S. Pat. No. 5,057,540. Derivatives of Quil™ A, for example QS21 (an HPLC purified fraction derivative of Quil™ A also known as QA21), and other fractions such as QA 17 are also disclosed. Semi-synthetic and synthetic derivatives of Quillaja Saponaria Molina saponins are also useful, such as those described in U.S. Pat. No. 5,977,081 and U.S. Pat. No. 6,080,725. The adjuvant denominated MF59 available from Chiron Corp. is described in U.S. Pat. No. 5,709,879 and U.S. Pat. No. 6,086,901.

Muramyl dipeptide adjuvants are also contemplated and include N-acetyl-muramyl-L-threonyl-D-isoglutamine (thur-MDP), N-acetyl-nor-muramyl-L-alanyl-D-isoglutamine [CGP 11637, referred to as nor-MDP], and N-acetylmuramyl-L-alanyl-D-isoglutaminyl-L-alanine-2-(1′-2′-dipalmityol-s-n-glycero-3-hydroxyphosphoryloxy) ethylamine [(CGP) 1983A, referred to as MTP-PE]. The so-called muramyl dipeptide analogues are described in U.S. Pat. No. 4,767,842.

Other adjuvant mixtures include combinations of 3D-MPL and QS21 (EP 0 671 948 B1), oil-in-water emulsions comprising 3D-MPL and QS21 (WO 95/17210, PCT/EP98/05714), 3D-MPL formulated with other carriers (EP 0 689 454 B1), QS21 formulated in cholesterol-containing liposomes (WO 96/33739), or immunostimulatory oligonucleotides (WO 96/02555). Adjuvant SBAS2 (now ASO₂) contains QS21 and MPL in an oil-in-water emulsion is also useful. Alternative adjuvants include those described in WO 99/52549 and non-particulate suspensions of polyoxyethylene ether (UK Patent Application No. 9807805.8).

The use of an adjuvant that contains one or more agonists for toll-like receptor-4 (TLR-4) such as an MPL® adjuvant or a structurally related compound such as an RC529® adjuvant or a Lipid A mimetic, alone or along with an agonist for TLR-9 such as a non-methylated oligo deoxynucleotide-containing the CpG motif is also optional.

Another type of adjuvant mixture comprises a stable water-in-oil emulsion further containing aminoalkyl glucosamine phosphates such as described in U.S. Pat. No. 6,113,918. Of the aminoalkyl glucosamine phosphates the molecule known as RC-529 {(2-[(R)-3-tetradecanoyloxytetradecanoylamino]ethyl 2-deoxy-4-O-phosphono-3-O—[(R)-3-tetradecanoyloxy-tetradecanoyl]-2-[(R)-3-tetradecanoyloxytetra-decanoylamino]-p-D-glucopyranoside triethylammonium salt.)} is the most preferred. A preferred water-in-oil emulsion is described in WO 99/56776.

Adjuvants are utilized in an adjuvant amount, which can vary with the adjuvant, host animal and immunogen. Typical amounts can vary from about 1 μg to about 1 mg per immunization. Those skilled in the art know that appropriate concentrations or amounts can be readily determined.

Vaccine compositions comprising an adjuvant based on oil in water emulsion is also included within the scope of the present invention. The water in oil emulsion may comprise a metabolisable oil and a saponin, such as for example as described in U.S. Pat. No. 7,323,182.

According to several embodiments, the vaccine compositions according to the present invention may contain one or more adjuvants, characterized in that it is present as a solution or emulsion which is substantially free from inorganic salt ions, wherein said solution or emulsion contains one or more water soluble or water-emulsifiable substances which is capable of making the vaccine isotonic or hypotonic. The water soluble or water-emulsifiable substances may be, for example, selected from the group consisting of: maltose; fructose; galactose; saccharose; sugar alcohol; lipid; and combinations thereof.

The GtS polypeptide fragments of the present invention comprise according to several specific embodiments a proteosome adjuvant. The proteosome adjuvant comprises a purified preparation of outer membrane proteins of meningococci and similar preparations from other bacteria. These proteins are highly hydrophobic, reflecting their role as transmembrane proteins and porins. Due to their hydrophobic protein-protein interactions, when appropriately isolated, the proteins form multi-molecular structures consisting of about 60-100 nm diameter whole or fragmented membrane vesicles. This liposome-like physical state allows the proteosome adjuvant to act as a protein carrier and also to act as an adjuvant.

The use of proteosome adjuvant has been described in the prior art and is reviewed by Lowell GH in “New Generation Vaccines”, Second Edition, Marcel Dekker Inc, New York, Basel, Hong Kong (1997) pages 193-206. Proteosome adjuvant vesicles are described as comparable in size to certain viruses which are hydrophobic and safe for human use. The review describes formulation of compositions comprising non-covalent complexes between various antigens and proteosome adjuvant vesicles which are formed when solubilizing detergent is selectably removed using exhaustive dialysis technology.

Vaccine compositions comprising different GtS fragments can be produced by mixing or linking a number of different GtS polypeptide fragments according to the invention with or without an adjuvant. In addition, GtS fragments according to the present invention may be included in a vaccine composition comprising any other S. pneumoniae protein or protein fragment, including mutated proteins such as detoxified pneumolysin, or they can be linked to or produced in conjunction with any such S. pneumoniae protein or protein fragment.

Vaccine compositions according to the present invention may include, for example, influenza polypeptides or peptide epitopes, conjugated with or coupled to at least one GtS polypeptide fragment according to the invention.

The antigen content is best defined by the biological effect it provokes. Naturally, sufficient antigen should be present to provoke the production of measurable amounts of protective antibody. A convenient test for the biological activity of an antigen involves the ability of the antigenic material undergoing testing to deplete a known positive antiserum of its protective antibody. The result is reported in the negative log of the LD₅₀ (lethal dose, 50%) for mice treated with virulent organisms which are pretreated with a known antiserum which itself was pretreated with various dilutions of the antigenic material being evaluated. A high value is therefore reflective of a high content of antigenic material which has tied up the antibodies in the known antiserum thus reducing or eliminating the effect of the antiserum on the virulent organism making a small dose lethal. It is preferred that the antigenic material present in the final formulation is at a level sufficient to increase the negative log of LD₅₀ by at least 1 preferably 1.4 compared to the result from the virulent organism treated with untreated antiserum. The absolute values obtained for the antiserum control and suitable vaccine material are, of course, dependent on the virulent organism and antiserum standards selected.

The following method may be also used to achieve the ideal vaccine formulation: starting from a defined antigen, which is intended to provoke the desired immune response, in a first step an adjuvant matched to the antigen is found, as described in the specialist literature, particularly in WO 97/30721. In a next step the vaccine is optimized by adding various isotonic-making substances as defined in the present inventions, preferably sugars and/or sugar alcohols, in an isotonic or slightly hypotonic concentration, to the mixture of antigen and adjuvant, with the composition otherwise being identical, and adjusting the solution to a physiological pH in the range from pH 4.0 to 10.0, particularly 7.4. Then, in a first step the substances or the concentration thereof which will improve the solubility of the antigen/adjuvant composition compared with a conventional, saline-buffered solution are determined. The improvement in the solubility characteristics by a candidate substance is a first indication that this substance is capable of bringing about an increase in the immunogenic activity of the vaccine.

Since one of the possible prerequisites for an increase in the cellular immune response is increased binding of the antigen to APCs (antigen presenting cells), in a next step an investigation can be made to see whether the substance leads to an increase of this kind. The procedure used may be analogous to that described in the definition of the adjuvant, e.g. incubating APCs with fluorescence-labelled peptide or protein, adjuvant and isotonic-making substance. An increased uptake or binding of the peptide to APCs brought about by the substance can be determined by comparison with cells which have been mixed with peptide and adjuvant alone or with a peptide/adjuvant composition which is present in conventional saline buffer solution, using throughflow cytometry.

The efficiency of the formulation may optionally also be demonstrated by the cellular immune response by detecting a “delayed-type hypersensitivity” (DTH) reaction in immunized animals.

Finally, the immunomodulatory activity of the formulation is measured in animal tests.

### Synthetic Peptides

The GtS polypeptide fragments of the present invention may be synthesized chemically using methods known in the art for synthesis of peptides and polypeptides. These methods generally rely on the known principles of peptide synthesis; most conveniently, the procedures can be performed according to the known principles of solid phase peptide synthesis.

As used herein “peptide” indicates a sequence of amino acids linked by peptide bonds. A polypeptide is generally a peptide of about 30 and more amino acids.

Polypeptide analogs and mimetics are also included within the scope of the invention as well as salts and esters of the polypeptides of the invention are encompassed. A polypeptide analog according to the present invention may optionally comprise at least one non-natural amino acid and/or at least one blocking group at either the C terminus or N terminus. Salts of the peptides of the invention are physiologically acceptable organic and inorganic salts. The design of appropriate “analogs” may be computer assisted.

The term “mimetic” means that a polypeptide according to the invention is modified in such a way that it includes at least one non-peptidic bond such as, for example, urea bond, carbamate bond, sulfonamide bond, hydrazine bond, or any other covalent bond. The design of appropriate “mimetic” may be computer assisted.

Salts and esters of the peptides of the invention are encompassed within the scope of the invention. Salts of the polypeptides of the invention are physiologically acceptable organic and inorganic salts. Functional derivatives of the polypeptides of the invention covers derivatives which may be prepared from the functional groups which occur as side chains on the residues or the N- or C-terminal groups, by means known in the art, and are included in the invention as long as they remain pharmaceutically acceptable, i.e., they do not destroy the activity of the polypeptide and do not confer toxic properties on compositions containing it. These derivatives may, for example, include aliphatic esters of the carboxyl groups, amides of the carboxyl groups produced by reaction with ammonia or with primary or secondary amines, N-acyl derivatives of free amino groups of the amino acid residues formed by reaction with acyl moieties (e.g., alkanoyl or carbocyclic aroyl groups) or O-acyl derivatives of free hydroxyl group (for example that of seryl or threonyl residues) formed by reaction with acyl moieties.

The term “amino acid” refers to compounds, which have an amino group and a carboxylic acid group, preferably in a 1,2-1,3-, or 1,4-substitution pattern on a carbon backbone. α-Amino acids are most preferred, and include the 20 natural amino acids (which are L-amino acids except for glycine) which are found in proteins, the corresponding D-amino acids, the corresponding N-methyl amino acids, side chain modified amino acids, the biosynthetically available amino acids which are not found in proteins (e.g., 4-hydroxy-proline, 5-hydroxy-lysine, citrulline, ornithine, canavanine, djenkolic acid, β-cyanolanine), and synthetically derived α-amino acids, such as amino-isobutyric acid, norleucine, norvaline, homocysteine and homoserine. β-Alanine and γ-amino butyric acid are examples of 1,3 and 1,4-amino acids, respectively, and many others are well known to the art. Statine-like isosteres (a dipeptide comprising two amino acids wherein the CONH linkage is replaced by a CHOH), hydroxyethylene isosteres (a dipeptide comprising two amino acids wherein the CONH linkage is replaced by a CHOHCH₂), reduced amide isosteres (a dipeptide comprising two amino acids wherein the CONH linkage is replaced by a CH₂NH linkage) and thioamide isosteres (a dipeptide comprising two amino acids wherein the CONH linkage is replaced by a CSNH linkage) are also useful residues for this invention.

The amino acids used in this invention are those, which are available commercially or are available by routine synthetic methods. Certain residues may require special methods for incorporation into the polypeptide, and sequential, divergent or convergent synthetic approaches to the peptide sequence are useful in this invention. Natural coded amino acids and their derivatives are represented by three-letter codes according to IUPAC conventions. When there is no indication, the L isomer was used.

Conservative substitutions of amino acids as known to those skilled in the art are within the scope of the present invention, as long as antigenicity is preserved in the substituted polypeptide. Conservative amino acid substitutions includes replacement of one amino acid with another having the same type of functional group or side chain e.g. aliphatic, aromatic, positively charged, negatively charged. These substitutions may enhance oral bioavailability, penetration into the central nervous system, targeting to specific cell populations and the like. One of skill will recognize that individual substitutions, deletions or additions to peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

### 1) Alanine (A), Serine (S), Threonine (T);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1

### A GtS Fragment

The amino acid sequence of S. pneumoniae GtS from serotype 4 TIGR4 strain (accession code NP_{—}346492,) is presented by SEQ ID NO:1:

```
    1 MSKDIRVRYA PSPTGLLHIG NARTALFNYL YARHHGGTFL IRIEDTDRKR HVEDGERSQL
  61 ENLRWLGMDW DESPESHENY RQSERLDLYQ KYIDQLLAEG KAYKSYVTEE ELAAERERQE
121 VAGETPRYIN EYLGMSEEEK AAYIAEREAA GIIPTVRLAV NESGIYKWHD MVKGDIEFEG
181 GNIGGDWVIQ KKDGYPTYNF AVVIDDHDMQ ISHVIRGDDH IANTPKQLMV YEALGWEAPE
241 FGHMTLIINS ETGKKLSKRD TNTLQFIEDY RKKGYLPEAV FNFIALLGWN PGGEDEIFSR
301 EEFIKLFDEN RLSKSPAAFD QKKLDWMSND YIKNADLETI FEMAKPFLEE AGRLTDKAEK
361 LVELYKPQMK SVDEIIPLTD LFFSDFPELT EAEREVMTGE TVPTVLEAFK AKLEAMTDDE
421 FVTENIFPQI KAVQKETGIK GKNLFMPIRI AVSGEMHGPE LPDTIFLLGR EKSIQHIENM
481 LKEISK
```

A fragment of the above protein lacking the N-terminal amino acids 1-332 amino acids was produces. The fragment denoted GtS (333-486), containing 154 amino acids corresponding to residues 333-486 of SEQ ID NO:1 is presented by SEQ ID NO:3:

MKNADLETIFEMAKPFLEEAGRLTDKAEKLVELYKPQMKSVDEIIPL
TDLFFSDFPELTEAEREVMTGETVPTVLEAFKAKLEAMTDDKFVTEN
IFPQIKAVQKETGIKGKNLFMPIRIAVSGEMHGPELPDTIFLLGREK
SIQHIENMLKEISK.

The nucleotides sequence of the fragment is presented by SEQ ID NO:11:

AAG AAT GCA GAC CTT GAA ACC ATC TTT GAA ATG GCA
AAA CCA TTC TTA GAG GAA GCA GGC CGT TTG ACT GAC
AAG GCT GAA AAA TTA GTT GAG CTC TAT AAA CCA CAA
ATG AAA TCA GTA GAT GAG ATT ATC CCA TTG ACA GAT
CTT TTC TTC TCA GAT TTC CCA GAA TTG ACA GAA GCA
GAG CGC GAA GTC ATG ACG GGT GAA ACA GTT CCA ACA
GTT CTT GAA GCA TTC AAA GCA AAA CTT GAA GCG ATG
ACA GAT GAT AAA TTT GTG ACA GAA AAT ATC TTC CCA
CAA ATT AAA GCA GTT CAA AAA GAA ACA GGT ATT AAA
GGG AAA AAT CTT TTC ATG CCT ATT CGT ATC GCA GTT
TCA GGC GAA ATG CAT GGG CCA GAA TTA CCA GAT ACA
ATT TTC TTG CTT GGA CGT GAA AAA TCA ATT CAG CAT
ATC GAA AAC ATG CTA AAA GAA ATC TCT AAA TAA.

### Example 2

### Homology to Human

A homology test comparing the amino acid sequence of the GtS (333-486) fragment of SEQ ID NO:3 with the human genome sequences was performed using http://blast.ncbi.nlm.nih.gov/Blast.cgi.

The highest homology found was between the S. pneumonia GtS fragment and the human protein glutamyl-tRNA synthetase 2 (Human GtS-2, GENE ID: 124454 EARS2, SEQ ID NO:12). The sequence identity between the intact S. pneumonia GtS protein sequence (SEQ ID NO:1) and the human GtS-2 protein (SEQ ID NO:12) is 29%. The sequence identity between the S. pneumonia GtS fragment 333-486 and the human intact GtS-2, (comparing SEQ ID NO:2 to SEQ ID NO:12) is 7.66%, while the sequence identity between the GtS fragment 333-486 (SEQ ID NO:2) and the corresponding amino acid residues of the human GtS-2 sequence (residues 361-521 of SEQ ID NO:12) is 18%. The N-terminal fragment of S. pneumonia GtS (residues 5-332 of SEQ ID NO:1) has 37% sequence identity to the corresponding amino acids of human GtS-2 protein (SEQ ID NO:12).

Clearly, the GtS polypeptide fragment of SEQ ID NO:2 has significant less sequence identity to human proteins than the intact S. pneumoniae GtS protein.

### Example 3

### Homology to Different S. pneumoniae Strains

The NCBI-Blast tool, was used to check the homology between the GtS (333-486) fragment of SEQ ID NO:3 and other S. pneumoniae strains. As demonstrated in table 1, all S. pneumoniae strains tested have at least 98% identity to SEQ ID NO:3, and 100% identity to SEQ ID NO:2 (in the relevant regions).

TABLE 1

Sequence identity

| S. pneumoniae strain | to SEQ ID NO: 3 | to SEQ ID NO: 2 |
|---|---|---|
| SP14-BS69 | 100% | 100% |
| Hungary19A-6 | 100% | 100% |
| SP23-BS72 | 100% | 100% |
| SP6-BS73 | 100% | 100% |
| R6 | 100% | 100% |
| D39 | 100% | 100% |
| SP18-BS74 | 99% | 100% |
| G54 | 99% | 100% |
| TIGR4 | 99% | 100% |
| SP11-BS70 | 99% | 100% |
| MLV-016 | 99% | 100% |
| CDC1087-00 | 99% | 100% |
| SP19-BS75 | 99% | 100% |
| CDC0288-04 | 99% | 100% |
| CDC3059-06 | 98% | 100% |
| CGSP14 | 98% | 100% |
| SP195 | 98% | 100% |
| SP9-BS68 | 98% | 100% |
| SP3-BS71 | 98% | 100% |
| CDC1873-00 | 98% | 100% |

The sequence mutations founds between the strains (maximum two differences per each two strains) are: L/F 382, G/D 400, K/E 421, I/V 466, and M/1481 (numbered according to SEQ ID NO:1).

### Example 4

### Cloning and Purification of the GtS Fragment

Cloning and purification of the GtS fragment were performed as described in Mizrachi-Nebenzahl et al. 2007, J Infect Dis. 196:945-53.

The GtS fragment was amplified from S. pneumoniae strain R6 genomic DNA by PCR using the following primers which contained Xohl and EcoRI recognition sequences, respectively:

Forward
(SEQ ID NO: 13)
5′GGAATTCAAGAATGCAGACCTTGAAACC 3′
Reverse
(SEQ ID NO: 14)
5′CCGCTCGAGTTATTTAGAGATTTCTTTTAGCAT 3′

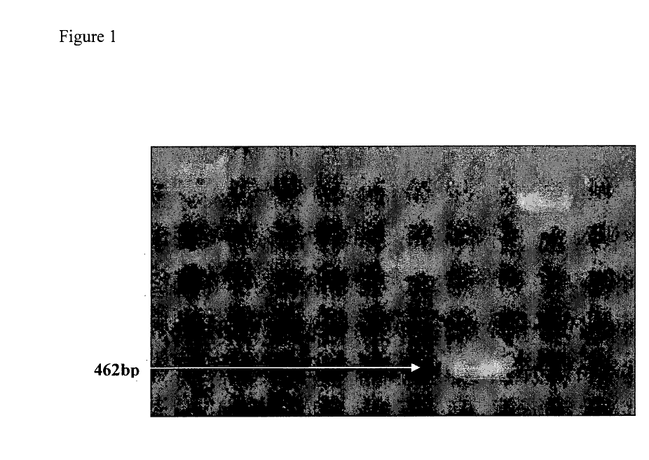

FIG. 1 represents amplification PCR of GtS (333-486) by genomic DNA.

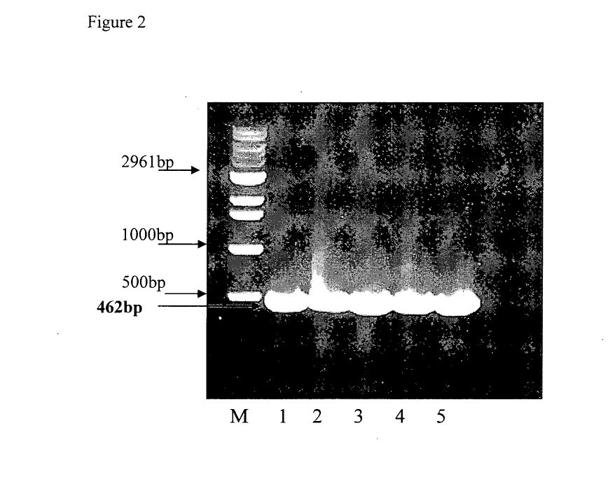

The amplified and Xohl-E.coRI (Takara Bio Inc, Shiga, Japan) digested DNA-fragments were cloned into the pET32a expression vector (BD Biosciences Clontech, Palo Alto, Calif., USA) and transformed in DH5a UltraMAX ultracompetent E. coli cells (Invitrogen, Carlsbad, Calif., USA). Ampicillin-resistant transformants were cultured and plasmid DNA was analyzed by PCR. The existence of the expected 462 bp size insert was confirmed by PCR amplification as shown in FIG. 2.

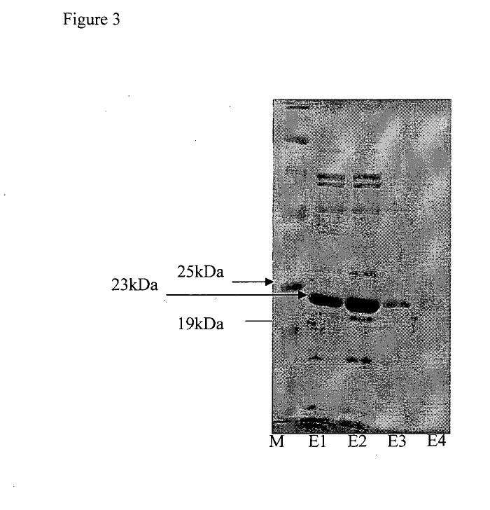

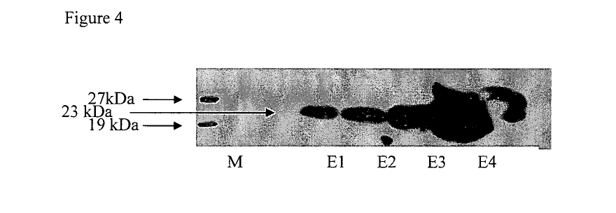

The modified (minus thioredoxin (TRX)) pET32a-GtS fragment vector was purified from DH5α UltraMAX cells using Qiagen High Speed Plasmid Maxi Kit (Qiagen GMBH, Hilden, Germany) and transformed in E. coli host expression strain BL21(DE3) pLysS (Stratagene, La Jolla, Calif.). The identity of the insert was confirmed by sequencing. Bacteria were grown over night and expression of the recombinant protein was induced by the addition of 1 mM IPTG to BL21(DE3) pLysS+6PGD cells for 5 hours. The cells were harvested by centrifugation, and lysed in lysis buffer. The HIS-tagged recombinant protein was purified using a Ni-NTA column (Qiagen GMBH, Hilden, Germany); binding for 1 hour at room temperature then the column was washed with wash buffer (8 M urea, 0.1 M NaH2PO₄, 0.01 M Tris-Cl pH 6.3), and the recombinant protein was recovered from the column using elution buffer (8 M urea, 0.1 M NaH₂PO₄, 0.01 M Tris-Cl, pH 5.9). Isolation of the protein was confirmed by Coomassie Brilliant blue staining and by Western blot analysis using anti-HIS antibodies (BD Biosciences Clontech, Palo Alto, Calif., USA). Resolution of the eluted protein by 1D-PAGE revealed a single band following staining with Coomassie Brilliant Blue (23 kDa band) as presented in FIG. 3. FIG. 4 represents western blot analysis 1D-PAGE using anti-HAT antibodies of the recombinant protein confirmed the 23 k Da band to be HIS-tagged-rGtS (333-486) fusion protein. An alternative approach is cloning the gene into pET30+vector omitting the His-tag sequence by the use of NdeI restriction enzyme to produce the first metionine. The DNA sequence optimized to E. coli codon usage of GtS fragment (333-486) including addition of terminal ATG (encoding Met residue), and a TAA stop codon was subcloned to pET 30+ to produce the actual untagged GTS fragment and is represented by SEQ ID NO:15:

ATGAAAAACGCTGATCTGGAAACTATTTTTGAAATGGCAAAACCGTTT
CTGGAAGAAGCAGGTCGTCTGACTGACAAAGCAGAGAAACTGGTTGAG
CTGTACAAACCGCAGATGAAATCTGTTGACGAGATCATTCCGCTGACT
GACCTGTTCTTTTCTGATTTCCCGGAACTGACTGAAGCAGAACGTGAA
GTAATGACTGGTGAAACTGTTCCGACTGTTCTGGAAGCGTTCAAAGCT
AAACTGGAGGCTATGACCGACGATAAATTCGTCACCGAAAACATCTTT
CCGCAGATCAAAGCGGTTCAGAAAGAAACCGGTATCAAAGGCAAAAAC
CTGTTCATGCCGATTCGTATTGCAGTATCTGGTGAAATGCATGGTCCG
GAACTGCCGGATACTATCTTTCTGCTGGGTCGTGAGAAATCTATCCAG
CACATTGAGAACATGCTGAAAGAGATCTCCAAATAA.

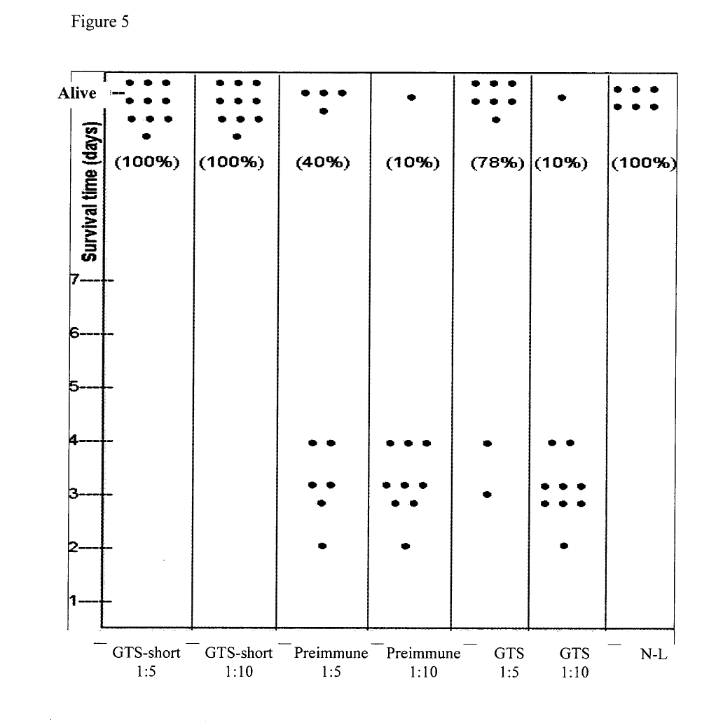

The produced polypeptide fragment was purified using three steps: ppt with AmSO₄, Q-sepharose, and two cycles over G-200 preparative chromatography column. The results were checked by An SDS-PAGE and FIG. 5 represent the untagged GtS 333-486 fragment (sGtS) from three consecutive tubes collected from the first cycle of the G-200 preparative column (out of fifteen columns runs reproducing similar results).

### In Vivo Models:

Following immunization with synthetic or recombinant GtS (333-486) derived from serotype 4 TIGR4 strain sequence, the animals are challenged with serotype 3 strain WU2. Additional experiment are performed to test the ability of this and other fragments to protect against additional S. pneumoniae strains which are serologically and genetically different from either serotype 4 strain TIGR 4 or serotype 3 strain WU2.

### Example 5

### Ex-Vivo Immunization with Rabbit Anti GtS (333-486) Antiserum

Two hundred CFU of S. pneumoniae strain 3 (WU2) were ex-vivo neutralized with rabbit anti GtS (333-486) and rabbit anti GtS diluted serums (1:5 and 1:10) for 1 hr and used to challenge 7 week old BALB/c female mice (n=10). Negative control mice (n=10) were challenged with 200 CFU of S. pneumoniae strain 3 (WU2) after neutralization with pre-immune diluted serums (1:5 and 1:10) obtained from the same rabbit. Positive control mice (n=10) were challenged with 200 CFU of S. pneumoniae strain 3 (WU2) after neutralization with rabbit anti Non-lectins serum. Survival was monitored for seven days.

The results depicted in FIG. 5 demonstrate 100 and 40% survival of mice after treatment with 1:5 and 1:10 anti GtS (333-486) diluted sera, respectively, while intact anti GtS diluted sera at 1:5 and 1:10 demonstrated only 78 and 10% survival, respectively.

It was therefore demonstrated that rabbit anti GtS (333-486) serum protected mice significantly (p<0.05) from an intraperitoneal lethal challenge with S. pneumoniae WU2.

### Example 6

### Vaccination Potential of rGtS Fragment in Mouse Models for Systemic Infections

For systemic S. pneumoniae lethal challenge mice immunized with rGtS fragment formulated with adjuvant and with adjuvant alone, as control, are inoculated intraperitoneally (i.p.) or intravenously (i.v) with a lethal dose of S. pneumoniae serotype 3 strain WU2. The inoculum's size is determined to be the lowest that cause 100% mortality in the control mice within 96-120 hours. Survival is monitored daily.

### Example 7

### Vaccination Potential of rGtS Fragment in Mouse Models for Upper Respiratory Lethal Infections

For respiratory S. pneumoniae lethal challenge mice immunized with rGtS fragment in adjuvant, and with adjuvant alone as control, are anaesthetized with isoflurane, and inoculated intranasally with a lethal dose of S. pneumoniae serotype 3 strain WU2 (in 25 μl PBS). The inoculum's size is determined to be the lowest that causes 100% mortality in the control mice within 96-120 hours. Survival is monitored daily.

In addition, the ability of immunization with GtS (333-486) to reduce S. pneumoniae bacterial load in the nasopharynx and prevention of aspiration to the lungs is tested.

### Example 8

### The Ability Antiserum Specific to GtS Fragments and of GtS Fragment to Inhibit Nasopharyngeal and Lung Colonization

To find whether GtS fragment is capable of inhibiting S. pneumoniae colonization, mice are inoculated intranasally with S. pneumoniae serotype 3 prior and after treatment ex vivo with antibodies to the GtS fragment. Alternatively, the GtS fragment, at concentrations ranging from 5-40 μg, is mixed with S. pneumoniae serotype 3, strain WU2 bacteria, and the mixture is inoculated intranasally with 5×10⁵ to 5×10⁷ S. pneumoniae. At 3, 6 24 and 48 hours following inoculation mice are sacrificed and the nasopharynx and lungs excised homogenized and plated onto blood agar plates for colony number enumeration.

### Example 9

### Otitis Media Models

Otitis media models in chinchilla and the rat (developed according to Chiavolini et al., 2008, Clinical Microbiology Reviews, 21:666-685; Giebink, G. S. 1999, Microb. Drug Resist., 5:57-72; Hermansson et al., 1988, Am. J. Otolaryngol. 9:97-101; and Ryan et al., 2006, Brain Res. 1091:3-8), are utilized to test the effectiveness of GtS fragments according to the invention. The ability of GtS (333-486) to protect those animal from developing otitis media following intranasal challenge is studied.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1.-25. (canceled)

26. A synthetic or recombinant polypeptide of 50-250 amino acids derived from the sequence of Streptococcus pneumonia (S. pneumoniae) glutamyl tRNA synthetase (GtS) of SEQ ID NO:1, comprising the sequence KNADLETIFEMAKPFLEEAGRLTDKAEKL (SEQ ID NO:2), and variants and analogs thereof.

27. The polypeptide according to claim 26 wherein the polypeptide consists of 100 to 200 amino acids or 130 to 180 amino acids.

28. The polypeptide according to claim 26 sharing less than 24% sequence identity with SEQ ID NO:12.

29. The polypeptide according to claim 26 sharing less than 10% sequence identity with SEQ ID NO:12.

30. The polypeptide according to claim 26 comprising the sequence:
(SEQ ID NO: 3)
XKNADLETIFEMAKPFLEEAGRLTDKAEKLVELYKPQMKSVDEIIPLT
DLFFSDFPELTEAEREVMTGETVPTVLEAFKAKLEAMTDDKFVTENIF
PQIKAVQKETGIKGKNLFMPIRIAVSGEMHGPELPDTIFLLGREKSIQ
HIENMLKEISK,
wherein X is Methionine or represents the polypeptide's N-terminus, and variants and analogs thereof.

31. The polypeptide according to claim 26 comprising the sequence:
(SEQ ID NO: 4)
XKNADLETIFEMAKPFLEEAGRLTDKAEKLVELYKPQMKSVDEIIPLT
DX₁FFSDFPELTEAEREVMTX₂ETVPTVLEAFKAKLEAMTDDX₃FVTE
NIFPQIKAVQKETGIKGKNLFMPIRIAVSGEMHGPELPDTX₄FLLGRE
KSIQHIENX₅LKEISK,
wherein X is Methionine or represents the polypeptide's N-terminus, X₁ is Leu (L) or Fhe (F), X₂ is Gly (G) or Asp (D), X₃ is Lys (K) or Glu (E), X₄ is Ile (I) or Val (V), and X₅ is Met (M) or Ile (I), and variants and analogs thereof.

32. The polypeptide according to claim 26 comprising a sequence selected from the group consisting of SEQ ID NOs: 5, 6, 7, 8, 9, and 10:
(SEQ ID NO: 5)
XKNADLETIFEMAKPFLEEAGRLTDKAEKLVELYKP;
(SEQ ID NO: 6)
MKNADLETIFEMAKPFLEEAGRLTDKAEKLVELYKPQMKSVDEIIPLT
DLFFSDFP;
(SEQ ID NO: 7)
XKNADLETIFEMAKPFLEEAGRLTDKAEKLVELYKPQMKSVDEIIPLT
DLFFSDFPELTEAEREVMTGETVPTVLEAFKAK;
(SEQ ID NO: 8)
MKNADLETIFEMAKPFLEEAGRLTDKAEKLVELYKPQMKSVDEIIPLT
DLFFSDEPELTEAEREVMTGETVPTVLEAFKAKLEAMTDDKEVTENIF
PQIKAVQKET;
(SEQ ID NO: 9)
XKNADLETIFEMAKPFLEEAGRLTDKAEKLVELYKPQMKSVDEIIPLT
DLFFSDFPELTEAEREVMTGETVPTVLEAFKAKLEAMTDDKFVTENIF
PQIKAVQKETGIKGKNLFMPIRIAVSG;
and
(SEQ ID NO: 10)
XKNADLETIFEMAKPFLEEAGRLTDKAEKLVELYKPQMKSVDEIIPLT
DLFFSDFPELTEAEREVMTGETVPTVLEAFKAKLEAMTDDKFVTENIF
PQIKAVQKETGIKGKNLEMPIRIAVSGEMHGPELPDTIFLLGR,
wherein X is Methionine or represents the polypeptide's N-terminus, and variants and analogs thereof.

33. The polypeptide according to claim 32 consisting of a sequence selected from the group consisting of SEQ ID NOs: 3, 4, 5, 6, 7, 8, 9 and 10.

34. The polypeptide according to claim 26 conjugated or fused to a carrier protein.

35. An isolated polynucleotide sequence encoding a polypeptide according to claim 26.

36. The isolated polynucleotide according to claim 35 encoding a polypeptide sequence selected from the group consisting of SEQ ID NOs: 3, 4, 5, 6, 7, 8, 9 and 10.

37. The isolated polynucleotide according to claim 35 comprising a sequence selected from the group consisting of SEQ ID NO:11 and SEQ ID NO:15.

38. The isolated polynucleotide according to claim 35 consisting of a sequence selected from the group consisting of SEQ ID NO:11 and SEQ ID NO:15.

39. A vaccine composition for immunization of a subject against S. pneumoniae comprising at least one polypeptide according to claim 26.

40. A vaccine composition for immunization of a subject against S. pneumoniae comprising at least two polypeptides according to claim 26.

41. The vaccine composition according to claim 40 further comprising an adjuvant.

42. The vaccine composition according to claim 41 wherein the adjuvant is selected from the group consisting of water in oil, emulsion, lipid emulsion, and liposome.

43. A method for inducing an immune response and conferring protection against S. pneumoniae in a subject, comprising administering to the subject a vaccine composition according to claim 39.

44. The method according to claim 43 wherein the route of administration of the vaccine is selected from intramuscular, intranasal, oral, intraperitoneal, subcutaneous, topical, intradermal, and transdermal delivery.

45. The method according to claim 44 wherein the vaccine composition is administered intramuscularly.
